(19) United States
(12) Patent Application Publication
(10) Pub. No.: US 2006/0264959 A1
(43) Pub. Date: Nov. 23, 2006

(54) Rod pusher

(75) Inventor: Mahmoud F. Abdelgany, Rockaway, NJ (US)

Correspondence Address: GIBB LP. LAW FIRM, LLC, 2568-A RIVA ROAD SUITE 304, ANNAPOLIS, MD, 21401, US

(73) Assignee: Custom Spine, Inc., Parsippany, NJ (US)

(21) Appl. No.: 11/217,032

(22) Filed: Aug. 31, 2005

Related U.S. Application Data
(60) Provisional application No. 60/683,734, filed on May 23, 2005.

(51) Int. Cl. A61F 5/00

(52) U.S. Cl. 606/86

(57) ABSTRACT

A rod pusher for engaging a rod for use in a spinal surgery, wherein the rod pusher comprises a shaft comprising a tip end and dual handle ends, wherein the dual handle end comprises a first handle receiver transverse to a second handle receiver, wherein the tip end comprises undercut grooves comprising one straight groove and at least one offset angled groove, wherein the first and second handle receivers each comprise indent and locking features to accommodate a handle used to apply force to the shaft, wherein the first handle receiver is positioned along a same longitudinal axis as the shaft, wherein the second handle is positioned along a transverse axis to the longitudinal axis of the shaft, and wherein the second handle is adapted to permit a user to rotate the shaft in reference to the rod thereby creating a fixable grip between the rod and the shaft.

### CROSS-REFERENCE TO RELATED APPLICATIONS

This application claims the benefit of U.S. Provisional Patent Application No. 60/683,734 filed on May 23, 2005, the contents of which, in its entirety, is herein incorporated by reference.

### BACKGROUND

1. Field of the Invention

The embodiments of the invention generally relate to medical devices, and, more particularly, to a medical device for use during spinal implant surgeries.

2. Description of the Related Art

Most conventional rod pushers 1, such as the one shown in FIG. 1, are simply shafts 2 with a round cutout 3 at one end with a handle 4 at the other end. These simple instruments tend to easily slip off a rod, which is used in spinal surgeries, potentially causing injury to the surrounding anatomy during alignment procedures. Furthermore, the handle may limit the visibility of the surgeon who is performing the operation. Typically, all the surgeon can do is push the rod down. However, usually the rod is not aligned on top of the bone screw and has to be slid medially or laterally, or even slightly pulled up potentially causing slippage problems. When slippage occurs, it is usually involves a violent motion/force. This is undesirable inside an open wound in a patient's back where sensitive anatomy may be exposed. Surgeons have identified that one of the most important aspects of performing spinal surgeries is having a good line of sight (i.e., visibility). Therefore, there remains a need for a novel rod pusher device capable of providing improved visibility for the surgeon during use.

### SUMMARY

In view of the foregoing, an embodiment of the invention provides a medical device for engaging a longitudinal member for use in a spinal surgery, wherein the medical device preferably comprises a shaft comprising a tip end and dual handle ends, wherein the dual handle end preferably comprises a first handle receiver transverse to a second handle receiver, wherein the tip end preferably comprises undercut grooves, the undercut grooves comprising one straight groove and at least one offset angled groove, wherein the first and second handle receivers each preferably comprise indent and locking features to accommodate a handle used to apply force to the shaft, wherein the first handle receiver is preferably positioned along a same longitudinal axis as the shaft, wherein the second handle is preferably positioned along a transverse axis to the longitudinal axis of the shaft, and wherein the second handle is preferably adapted to permit a user to rotate the shaft in reference to the longitudinal member thereby creating a fixable grip between the longitudinal member and the shaft. The tip end preferably comprises a plurality of arms spaced apart from one another. Moreover, the first and second handle receivers preferably form an acute angle with respect to the shaft.

These and other aspects of the embodiments of the invention will be better appreciated and understood when considered in conjunction with the following description and the accompanying drawings. It should be understood, however, that the following descriptions, while indicating preferred embodiments of the invention and numerous specific details thereof, are given by way of illustration and not of limitation. Many changes and modifications may be made within the scope of the embodiments of the invention without departing from the spirit thereof, and the embodiments of the invention include all such modifications.

### BRIEF DESCRIPTION OF THE DRAWINGS

The embodiments of the invention will be better understood from the following detailed description with reference to the drawings, in which:

FIG. 1 illustrates a schematic diagram of a conventional rod pusher;

FIGS. 2(A) through 2(F) illustrate schematic diagrams of a rod pusher according to an embodiment of the invention;

FIG. 3 illustrates a rod pusher attached to handles according to an embodiment of the invention; and

FIG. 4 illustrates an isolated view of one handle engaging the rod pusher shaft of the rod pusher of FIG. 3 according to an embodiment of the invention.

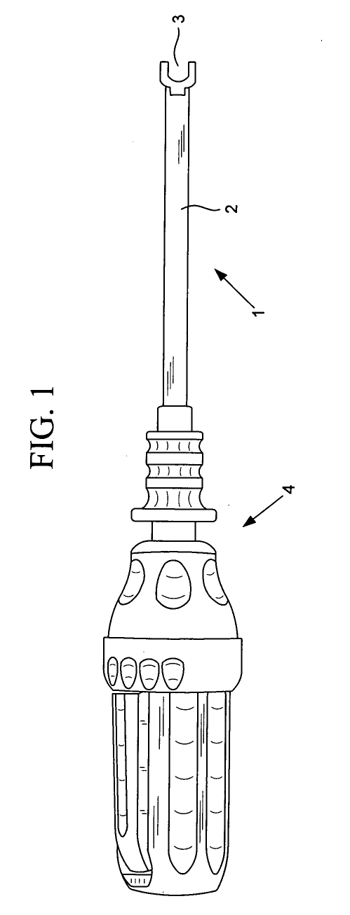

### DETAILED DESCRIPTION OF PREFERRED EMBODIMENTS

The embodiments of the invention and the various features and advantageous details thereof are explained more fully with reference to the non-limiting embodiments that are illustrated in the accompanying drawings and detailed in the following description. It should be noted that the features illustrated in the drawings are not necessarily drawn to scale. Descriptions of well-known components and processing techniques are omitted so as to not unnecessarily obscure the embodiments of the invention. The examples used herein are intended merely to facilitate an understanding of ways in which the embodiments of the invention may be practiced and to further enable those of skill in the art to practice the embodiments of the invention. Accordingly, the examples should not be construed as limiting the scope of the embodiments of the invention.

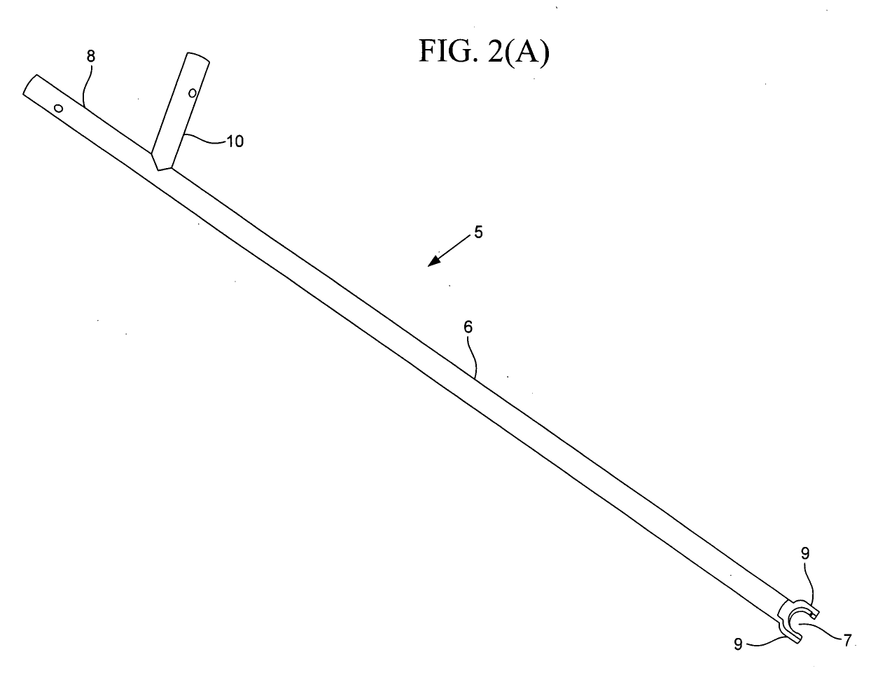

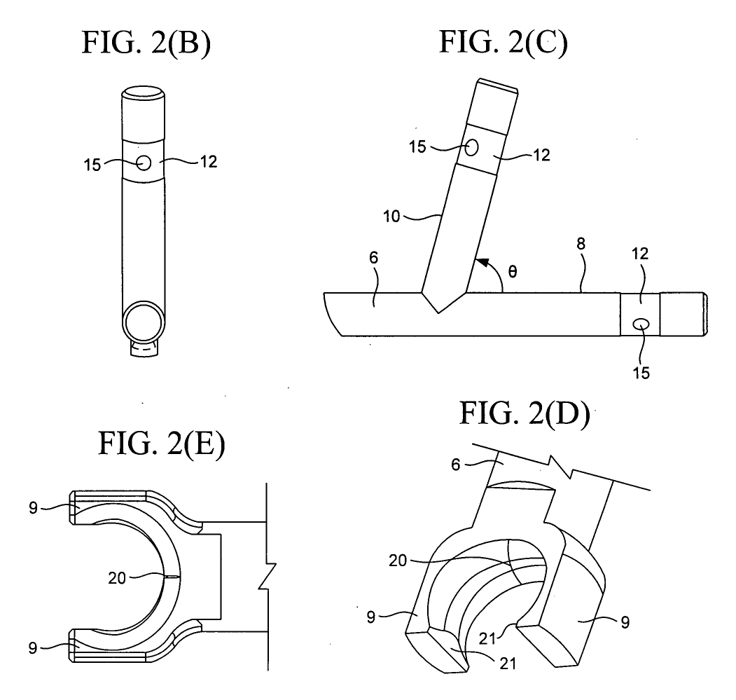

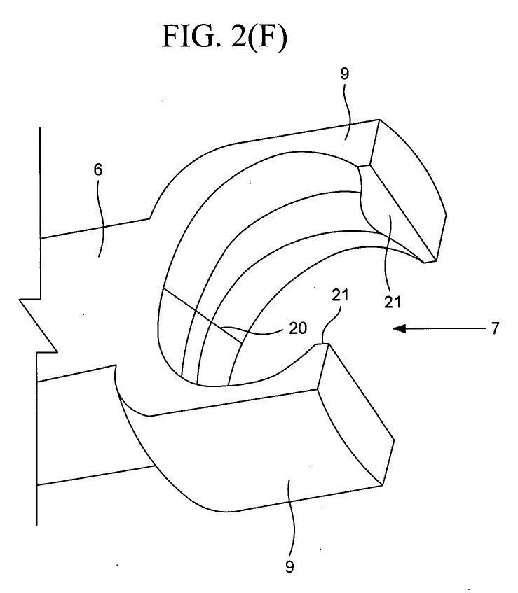

As mentioned, there remains a need for a novel rod pusher device capable of providing improved visibility for the surgeon during use. The embodiments of the invention achieve this by cutting the U-shaped channel of a rod pusher three times in the tip (once straight, then twice more at offset angles). Hence, the surgeon can get a very firm grip on the rod by simply twisting the rod pusher around the rod. The operator can then manipulate the rod in any direction while his/her hand is removed from the line of sight, thereby increasing surgical visibility. Referring now to the drawings, and more particularly to FIGS. 2(A) through 4, there are shown preferred embodiments of the invention.

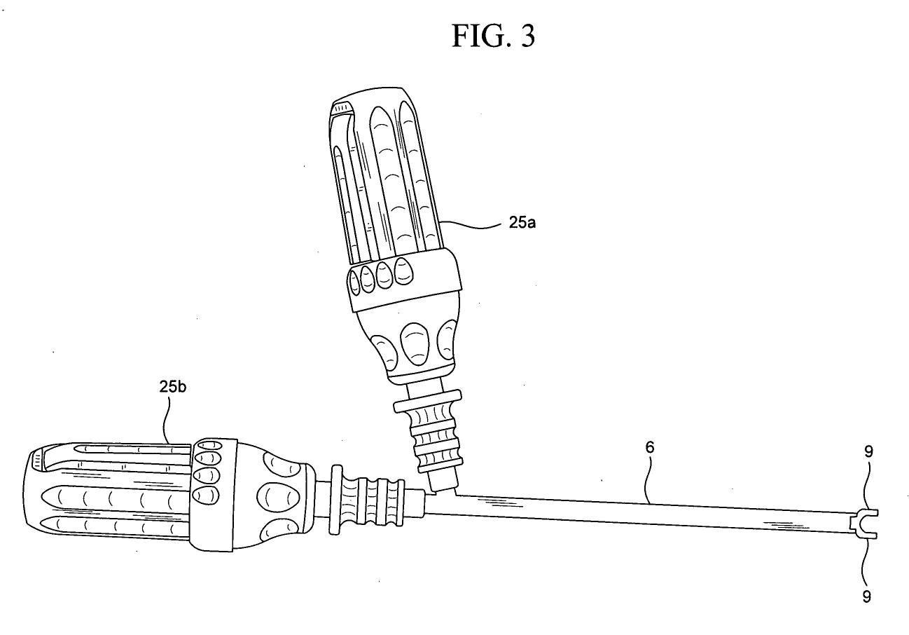

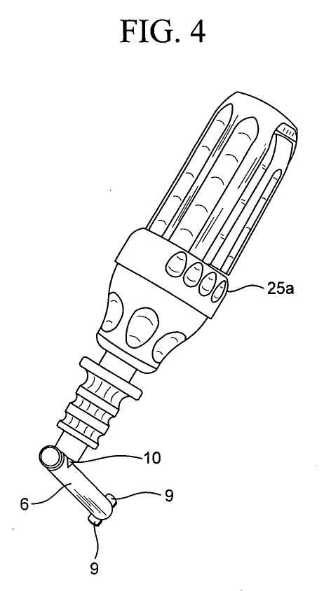

FIGS. 2(A) through 2(F) illustrate various views of components of a rod pusher device 5 according to an embodiment of the invention. Generally, the rod pusher shaft 6 includes a channel end 7 and dual gripping ends 8, 10 for accommodating a handle, such as a screwdriver-like handle described in U.S. patent application Ser. Nos. 11/063,452 and 11/076,670, the complete disclosures of which, in their entireties, are herein incorporated by reference. The channel end 7 comprises two arms 9 that together form into a U-shape. The channel end 7 is dimensioned and configured to receive a longitudinal member (i.e., rod) (not shown), such as the one described in U.S. patent application Ser. No. 11/045,908, the complete disclosure of which, in its entirety, is herein incorporated by reference, used in the spinal surgery. Specifically, the tip of the channel end 7 comprises undercut grooves 20, 21 configured therein; one straight groove 20 and two grooves 21 offset at angles with respect to the straight groove 20. Each of the gripping ends 8, 10 includes indent features 12 and holes 15 to accommodate a handle (shown in FIGS. 3 and 4), wherein the handle slides over one of the gripping ends 8, 10, locks into place in the indent features 12, and is retained by a pin (not shown), which engages the hole 15 of each of the gripping ends 8, 10.

FIG. 3 illustrates the rod pusher 5 according to an embodiment of the invention, wherein it is illustrated how dual handles 25a, 25b may be used in accordance with the rod pusher 5. Once in position, the dual handles 25a, 25b are transverse to one another and allow for rotation of the rod pusher shaft 6 in multi-planes. FIG. 4 illustrates an isolated view of one handle 25a engaging the rod pusher shaft 6 of the rod pusher 5 of FIG. 3 according to an embodiment of the invention.

The embodiments of the invention provide a multifunction rod pusher 5 to enable a surgeon to align, pull, slide, and push a spinal rod into a top loading bone anchor, such as the ones described in U.S. patent application Ser. No. 11/045,908, the complete disclosure of which, in its entirety, is herein incorporated by reference, during a spinal surgery. This is accomplished by “twisting” the instrument around a rod locking it into undercut grooves 20, 21 within the rod pusher tip 7 and providing a positive grip.

The rod pusher 5 is attached to one or two quick release handles 25a, 25b and is dropped on an unaligned rod (not shown) that has been secured to an adjacent bone screw (not shown), such as the ones described in U.S. patent application Ser. No. 11/045,908, the complete disclosure of which, in its entirety, is herein incorporated by reference. The handle 25a in the side position allows the operator to rotate the rod pusher 5 with reference to the rod creating a firm grip between the rod and rod pusher 5. The rod pusher 5 can now be used manipulate the rod in any direction without losing its grip because of the locking forces derived from the twisting of the rod pusher 5 onto the rod.

Generally, as illustrated in FIGS. 2(A) through 4, the embodiments of the invention provides a medical device 5 for engaging a longitudinal member (not shown) for use in a spinal surgery, wherein the medical device 5 preferably comprises a shaft 6 comprising a tip end 7 and dual handle ends 8, 10, wherein the dual handle end 8, 10 preferably comprises a first handle receiver 8 transverse to a second handle receiver 10, wherein the tip end 7 preferably comprises undercut grooves 20, 21. The undercut grooves 20, 21 comprise one straight groove 20 and at least one offset angled groove 21, wherein the first and second handle receivers 8, 10 each preferably comprise indent 15 and locking 12 features to accommodate a handle 25a, 25b used to apply force to the shaft 6, wherein the first handle receiver 8 is preferably positioned along a same longitudinal axis as the shaft 6, and wherein the second handle 10 is preferably positioned along a transverse axis to the longitudinal axis of the shaft 6. Preferably, the second handle 10 is preferably adapted to permit a user to rotate the shaft 6 in reference to the longitudinal member thereby creating a fixable grip between the longitudinal member and the shaft 6. The tip end 7 preferably comprises a plurality of arms 9 spaced apart from one another. Moreover, the first and second handle receivers 8, 10 preferably form an acute angle θ (shown in FIG. 2(C)) with respect to the shaft 6.

The foregoing description of the specific embodiments will so fully reveal the general nature of the invention that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments of the invention have been described in terms of preferred embodiments, those skilled in the art will recognize that the embodiments of the invention can be practiced with modification within the spirit and scope of the appended claims.

What is claimed is:

1. A medical device for engaging a longitudinal member for use in a spinal surgery, said medical device comprising:
a shaft comprising a tip end and dual handle ends, wherein the dual handle end comprises a first handle receiver transverse to a second handle receiver,
wherein said tip end comprises undercut grooves, said undercut grooves comprising one straight groove and at least one offset angled groove.

2. The medical device of claim 1, wherein the first and second handle receivers each comprise indent and locking features to accommodate a handle used to apply force to said shaft.

3. The medical device of claim 1, wherein said first handle receiver is positioned along a same longitudinal axis as said shaft.

4. The medical device of claim 1, wherein said second handle is positioned along a transverse axis to said longitudinal axis of said shaft.

5. The medical device of claim 1, wherein said second handle is adapted to permit a user to rotate said shaft in reference to said longitudinal member thereby creating a fixable grip between said longitudinal member and said shaft.

6. The medical device of claim 1, wherein said tip end comprises a plurality of arms spaced apart from one another.

7. The medical device of claim 1, wherein the first and second handle receivers each comprise indent and locking features.

8. The medical device of claim 1, wherein said second handle is adapted to permit rotation of said shaft in reference to said longitudinal member.

9. The medical device of claim 1, wherein the first and second handle receivers form an acute angle with respect to said shaft.

10. A medical device for engaging a longitudinal member for use in a spinal surgery, said medical device comprising:
a shaft comprising a tip end and dual handle ends, wherein the dual handle end comprises a first handle receiver transverse to a second handle receiver,
wherein said tip end comprises undercut grooves, said undercut grooves comprising one straight groove and at least one offset angled groove,
wherein said first handle receiver is positioned along a same longitudinal axis as said shaft, and
wherein said second handle is positioned along a transverse axis to said longitudinal axis of said shaft.

11. The medical device of claim 10, wherein the first and second handle receivers each comprise indent and locking features to accommodate a handle used to apply force to said shaft.

12. The medical device of claim 10, wherein said second handle is adapted to permit a user to rotate said shaft in reference to said longitudinal member thereby creating a fixable grip between said longitudinal member and said shaft.

13. The medical device of claim 10, wherein said tip end comprises a plurality of arms spaced apart from one another.

14. The medical device of claim 10, wherein the first and second handle receivers each comprise indent and locking features.

15. The medical device of claim 10, wherein said second handle is adapted to permit rotation of said shaft in reference to said longitudinal member.

16. The medical device of claim 10, wherein the first and second handle receivers form an acute angle with respect to said shaft.

17. A medical device for engaging a longitudinal member for use in a spinal surgery, said medical device comprising:
a shaft comprising a tip end and dual handle ends, wherein the dual handle end comprises a first handle receiver transverse to a second handle receiver,
wherein said tip end comprises undercut grooves, said undercut grooves comprising one straight groove and at least one offset angled groove,
wherein said tip end comprises a plurality of arms spaced apart from one another,
wherein said second handle is adapted to permit rotation of said shaft in reference to said longitudinal member thereby creating a fixable grip between said longitudinal member and said shaft, and
wherein the first and second handle receivers form an acute angle with respect to said shaft.

18. The medical device of claim 17, wherein the first and second handle receivers each comprise indent and locking features to accommodate a handle used to apply force to said shaft.

19. The medical device of claim 17, wherein said first handle receiver is positioned along a same longitudinal axis as said shaft.

20. The medical device of claim 17, wherein said second handle is positioned along a transverse axis to said longitudinal axis of said shaft.
